(19) United States
(12) Patent Application Publication
(10) Pub. No.: US 2006/0247733 A1
(43) Pub. Date: Nov. 2, 2006

(54) Garment for electrical muscle stimulation of muscles in the upper body and arms and legs

(76) Inventor: Salah Amer, Coral Gables, FL (US)

Correspondence Address: MICHAEL J. BUCHENHORNER, 8540 S.W. 83 STREET, MIAMI, FL, 33143, US

(21) Appl. No.: 11/120,060

(22) Filed: May 2, 2005

(51) Int. Cl. A61N 1/04; A61N 1/18

(52) U.S. Cl. 607/48; 607/149; 607/148

(57) ABSTRACT

A garment, for the upper body of a wearer, includes: a covering of the upper body including sleeves for covering substantially all of the wearer's arms; a plurality of electrodes distributed throughout the garment at locations where each electrode makes electrical contact with one or more muscles of a wearer; an electrical power source for providing an electrical current to the plurality of electrodes; a control device for controlling the amount of electrical current provided to the wearers muscles. The garment includes a material that provides a spring force on the plurality of electrodes such that electrical conductivity between the electrodes and the skin of the wearer is maintained while the garment is worn.

### FIELD OF THE INVENTION

The invention disclosed broadly relates to the field of electrical muscle simulation (EMS) and more particularly to a method and system for applying a controlled electric stimulus to muscles in the upper body muscles to promote caloric consumption.

### BACKGROUND OF THE INVENTION

Electrical muscle stimulation (EMS) is a known way to cause muscles to contract. This technology uses conductive pads or electrodes externally to apply a weak current to a muscle or group of muscles to cause them to contract. Each electrode receives an electric stimulation signal from an electric power source, such as an EMS machine. To date EMS has not been used to elicit a cardiovascular exercise effect in adults without joint loading.

Studies have shown that exercise of the upper body promote the consumption of calories or body fat. The use of EMS to elicit contraction of muscles has been done for prevention of muscle atrophy, muscle strengthening, management of incontinence, spinal deformities, and spasticity. It is also used to exercise muscles that are immobilized for long periods of time as a result of muscular or neurological disorders, or extended periods of bed rest arising from injury, surgery, or illness. EMS is also useful for the general exercise of functional muscles to improve muscle tone and strength. For example, athletes can use EMS to treat muscle injuries as a supplement to conventional conditioning exercises. EMS can also be used to recondition muscles or muscle groups which have, for whatever reason, lost their tone and/or strength, have been injured, or are in need of reconditioning to effect cosmetic improvements. An operator who has been trained in the principles of EMS can analyze the areas which are of concern and select the proper muscles to exercise and train.

The electrical stimulation signal can be adjusted in amplitude, polarity, frequency, waveform, and the like. U.S. Pat. No. 6,341,237 illustrates a device for administrating EMS which includes a flexible covering having a plurality of spaced apart electrodes. In that patent the flexible covering is shaped like a band or belt, and is designed to encircle and be connected around a portion of a patient's body. The band or belt is made from an elastic material so that the electrodes are pressed against the skin of the patient to promote better electrical conduction. Electrodes are selectively positionable to different locations on the flexible covering so they may be placed directly over a selected muscle or muscle group. Each electrode has its individual control for adjusting the level of the electrical stimulation signal so that different muscles can receive different levels of stimulation and the level of stimulation may be changed during the course of treatment. A master adjustment control can be used to adjust the stimulation signal level applied to all electrodes. In a preferred embodiment, the individual adjustment controls are located adjacent their respective electrodes on the flexible covering.

U.S. Pat. No. 4,480,830 illustrates a method and apparatus for exercising paralyzed muscles. The method and apparatus make use of a set of transcutaneous electrodes which are placed upon the skin of the subject over muscles which are to be stimulated. A computer controlled stimulator generates a pair of alternately pulsed stimulation signals which are applied across different pairs of stimulation electrodes to produce controlled muscle contraction. Muscle movement is resisted by a dynamic load and a position sensor provides a feedback signal indicating the movement actually achieved. The computer uses the feedback signal for modifying the control signal applied to the stimulator. U.S. Pat. No. 4,499,990 shows a system and method for treating persons with paralyzed legs. The apparatus and method include four sets of transcutaneous electrodes which are placed above the iliac and quadriceps muscles of the paralyzed person. The person is seated upon an exercise bicycle and a series of pulsed stimulation signals are applied to the electrodes to cause coordinated contraction of the iliac and quadriceps muscles. This causes pedaling of the exercise cycle by the paralyzed legs. A position sensor senses the position of the pedals and transmits an indication thereof to a computer which generates control signals for stimulation driving circuits connected to the stimulation electrodes.

U.S. Pat. No. 4,586,495 illustrates an apparatus and method for stimulating muscular activity in an acutely injured patient. A leg which is to be stimulated is strapped into a brace and the leg muscles are stimulated to work isometrically against the brace. The effort exerted by the muscles is measured by load cells which generate feedback signals for a control computer. The computer adjusts the stimulation signals in accordance with the received feedback signals.

U.S. Pat. No. 4,586,510 discloses an apparatus for exercising a paralyzed limb by functional electrical stimulation. The system utilizes simple analog devices including a reference signal generator, a position sensor, and an error signal generator. The error signal is integrated to produce a stimulation driving signal for application to the stimulation electrodes mounted on the limb. In the disclosed embodiment, the paralyzed person may be seated in an exercise chair which is equipped with a pair of loading assemblies which are attachable to the legs of the person so as to yieldingly resist the stimulated movement.

U.S. Pat. No. 4,724,842 shows a method and apparatus for muscle stimulation. An exercise machine or dynamometer is provided with control apparatus for ascertaining the physical position of a patient during an exercise. The patient is then electrically stimulated over selected ranges of motion in order to aid in the exercise.

U.S. Pat. No. 5,070,873 includes a method of and apparatus for electrically stimulating quadriceps muscles of an upper motor unit paraplegic. Muscle fatigue of an electrically stimulated quadriceps muscle of an upper motor neuron paraplegic is detected and compensated for by monitoring the myoelectric (EMG) signal produced by the stimulated muscle and controlling one or more of the following parameters of the electrical stimulation (ES) signal: pulse repetition rate, amplitude, and pulse width.

U.S. Pat. No. 5,507,788 illustrates a method and apparatus for controlling skeletal muscle fatigue during electrical stimulation. Electrical stimulation signals are applied to muscles at a frequency which is varied in response to a detected ripple signal in an output tension or torque record which corresponds to the fusion of the multiple muscle contractions. An average torque amplitude is first determined when a stimulation signal is applied at an initial frequency. The amplitude of the ripple on the torque output is then determined and compared to the average torque amplitude to provide a ripple percentage. The measured ripple percentage is compared to a selected ripple percentage corresponding to the desired fusion of the multiple muscle contractions. And the stimulation frequency is adjusted by a feedback loop until the measured ripple percentage conforms to the selected value.

U.S. Pat. No. 5,628,722 shows a method for maintaining knee stability of a user suffering from damage to a knee ligament. The method includes a sensor feedback system for measuring abnormal physical relationships between the tibia and femur. The sensor feedback system determines whether selected conditions have been met warranting the application of electrical stimulation and provides information regarding the determination to an electronic stimulator. Electrodes are mounted on the hamstring and/or quadriceps muscles in electrical communication with the electronic stimulator for causing contraction of the thigh muscles at selected levels, thus providing a posteriorly and/or anteriorly directed force to the upper tibial bone and thereby preventing its instability.

U.S. Pat. No. 6,876,883 relates to a belt comprising electrodes for simulating muscles during exercise. However, that approach ignores the value of upper body exercise and is useful only during physical exercise.

There thus is a need for a garment or suit to stimulate upper body muscles and that causes caloric consumption with or without exercise.

### SUMMARY OF THE INVENTION

Briefly, according to an embodiment of the invention, a garment, for the upper body of a wearer, includes: a covering of the upper body including sleeves for covering substantially all of the wearer's arms; a plurality of electrodes distributed throughout the garment at locations where each electrode makes electrical contact with one or more muscles of a wearer; an electrical power source for providing an electrical current to the plurality of electrodes; a control device for controlling the amount of electrical current provided to the wearers muscles. The garment comprises a material that provides a spring force on the plurality of electrodes such that electrical conductivity between the electrodes and the skin of the wearer is maintained while the garment is worn.

### BRIEF DESCRIPTION OF THE DRAWINGS

FIG. 1 is an illustration of a tight-fitting muscle-stimulating garment for the upper body according to an embodiment of the invention.

FIG. 2 is a flow chart of a method for electrical stimulation of muscles according to another embodiment of the invention.

FIG. 3 shows another embodiment of the invention wherein a body suit also includes a plurality of electrodes in the inside of the garment so that muscles throughout the body are stimulated into contraction to induce calorie consumption.

FIG. 4 shows another embodiment of the invention wherein trousers or pants also includes a plurality of electrodes in the inside of the garment so that muscles in the lower body are stimulated into contraction to induce calorie consumption.

### DETAILED DESCRIPTION

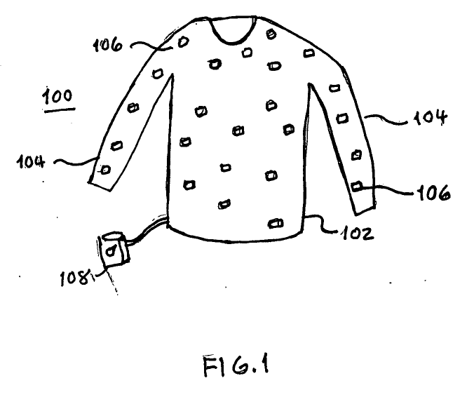

Referring to FIG. 1, there is shown a garment 100 for the upper body, according to an embodiment of the invention. The garment 100 comprises a first portion 102 that covers the torso of the wearer and a pair of long sleeves 104 that cover the arms of the wearer. The garment 100 also comprises a plurality of electrodes 106 attached to the inside of the garment 100 such that they come into electrically conductive contact with the skin of the user. Each electrode is positioned at locations where each electrode makes electrical contact with one or more muscles of a wearer. The garment should be tight fitting so that, when worn, the electrical contacts make good conductive contact with the wearer's skin. Moreover, the material of the garment should have a spring-like or elastic quality that keeps the electrical contact effective even though the wearer changes position of his or her body. Alternatively, the electrodes can include an adhesive that keeps the electrodes in electrical contact with the body (skin) of the wearer. It is particularly beneficial that electrodes 106 be included in the sleeves of the garment 100 such that the muscles in the arms contract when the garment is worn.

The garment 100 covers the upper body of the wearer and includes sleeves 108 for covering substantially all of the wearer's arms. The electrodes 106 in the sleeves 104 come into electrical contact with the wearer's skin such that the wearer's arm muscles are stimulated to contract. This contraction causes the muscles to consume fat by burning calories substantially the same as if the muscles were in dynamic exercise.

The garment 100 is preferably made form a non-conducting material such that the current provided by the electrodes 106 flows into the user instead of being conducted along the surface of the garment.

A control device 108, for controlling the amount of electrical current provided to the wearers muscles, is electrically connected to the electrodes 106 in the garment 100 via a plurality of wires (not shown). The wearer uses the control device to adjust the level of current applied to the muscles. The wearer will select a level of current that does not result in discomfort. Preferably, the wearer will select the highest level of current that does not result in discomfort. A stimulation protocol such as that described by Nuhr, “Beneficial Effects of Chronic Low Frequency Stimulation of Thigh Muscles in Patients with Advanced Chronic Heart Failure” can be used [see http://eurheart.org/cgi/content/full/25/2/136].

Thus, an adjustable intensity up to 100 milli-amperes for each channel of a stimulator can be used. Biphasic symmetric, constant voltage impulses with a purse width of 0.5 milliseconds and a frequency of 15 Hertz can also be used. Impulse trains can be delivered for two seconds and paused for four seconds. Such stimulation can be performed for several hours each day (e.g. two hours).

The control device 110 can include a display for providing feedback to the user on the level of current being applied and the duration or remaining time of the application of current to the electrodes. The electrodes can also include sensors (or separate sensors can be used) that monitor body conditions during the application of the EMS. The electrodes are typically of the kind that are used in electrocardiogram machines or similar to those described in U.S. Pat. No. 4,729,377, issued to Granek et al. in Mar. 8, 1988. The electrodes 106 can be of any appropriate type for conducting electricity to the wearer's body. An example of a suitable electrode is a self-adhering rectangular surface (130 cm² Value Trade® made by AXELGAARD Manufacturing Co., Ltd. Lystrup, Denmark).

The display can also provide the user an indication of the quality of the electrical contact between the electrode and the skin of the user.

An electrical power source 112 provides the electrical current to the plurality of electrodes that stimulates the wearer's muscles. The power source 112 can be a battery or a power cord with a transformer for connecting to a wall power outlet. The power supply must provide sufficient power to allow the user to provide sufficient electrical current to stimulate the wearer's muscles into a calorie consuming state.

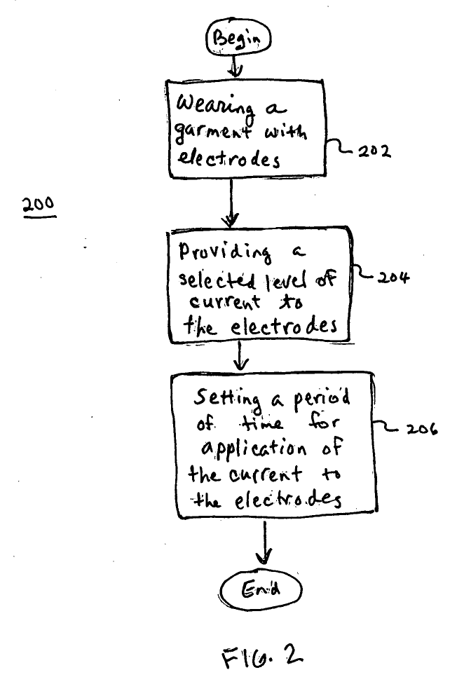

FIG. 2 is a flow chart of a method 200 for electrical stimulation of muscles according to another embodiment of the invention. In step 202 the user wears a garment with electrodes as described above. In step 204, the user applies an electric current to the electrodes such that selected muscles contract.

In step 206 the user selects a period of time during which the current will be applied. The control unit can be programmed to default to a duration that is minimally beneficial to the user. The method ends when the period of duration is over.

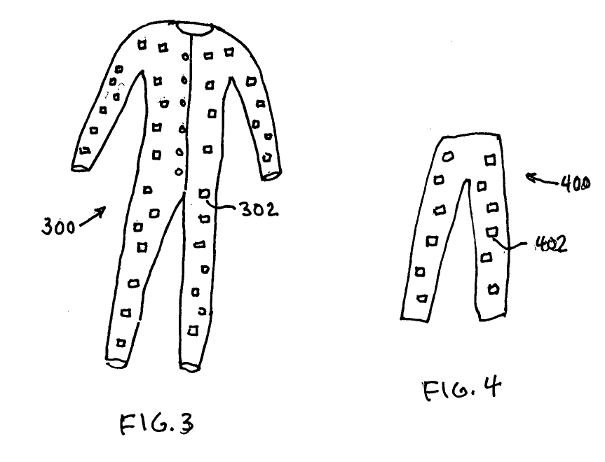

FIG. 3 shows another embodiment of the invention wherein a body suit 300 also includes a plurality of electrodes in the inside of the garment so that muscles throughout the body are stimulated into contraction to induce calorie consumption. FIG. 4 shows another embodiment of the invention wherein trousers or pants 400 also includes a plurality of electrodes in the inside of the garment so that muscles in the lower body are stimulated into contraction to induce calorie consumption.

Therefore, while there has been described what is presently considered to be the preferred embodiment, it will understood by those skilled in the art that other modifications can be made within the spirit of the invention.

We claim:

1. A garment, for stimulating muscles of a wearer, comprising:
a covering of the upper body including sleeves for covering substantially all of the wearer's arms;
a plurality of electrodes distributed throughout the garment at locations where each electrode makes electrical contact with one or more muscles of a wearer;
an electrical power source for providing an electrical current to the plurality of electrodes;
a control device for controlling the amount of electrical current provided to the wearers muscles;
wherein the garment comprises a material that provides a spring force on the plurality of electrodes such that electrical conductivity between the electrodes and the skin of the wearer is maintained while the garment is worn.

2. The garment of claim 1, wherein the control device includes a display for providing the wearer an indication of caloric consumption.

3. The garment of claim 1, wherein the electrical power source comprises a battery.

4. The garment of claim 1, wherein the electrical power source comprises a connection to an electrical outlet.

5. The garment of claim 1, wherein the control device provides a coupling between the electrodes and the electrical power source.

6. The garment of claim 1, wherein the control device comprises an interface for showing the wearer an indication of the calories being consumed.

7. The garment of claim 1, further comprising legs comprising further electrodes.

8. A method for stimulating upper body muscles of a person, the method comprising steps of:
wearing a garment covering of the upper body including sleeves for covering substantially all of the person's arms;
activating a plurality of electrodes distributed throughout the garment at locations where each electrode makes electrical contact with one or more muscles of the person such that the muscles contract while the current is applied;
wherein the garment comprises a material that provides a spring force on the plurality of electrodes such that electrical conductivity between the electrodes and the skin of the wearer is maintained while the garment is worn.

9. The method of claim 8 further comprising adjusting the amount of electrical current provided to the wearers muscles based on the comfort level experienced by the person.

10. The method of claim 8 further comprising providing feedback to the user indicating the amount of calories being consumed.

11. The method of claim 8 further comprising providing feedback to the user indicating the level of current being applied to the muscles.
